(19) United States
(12) Patent Application Publication
(10) Pub. No.: US 2015/0128956 A1
(43) Pub. Date: May 14, 2015

(54) NECK EXTENSION DEVICE AND METHOD OF USE

(71) Applicant: KONINKLIJKE PHILIPS N.V., EINDHOVEN (NL)

(72) Inventor: Jerome Matula, JR., Apollo, PA (US)

(21) Appl. No.: 14/406,552

(22) PCT Filed: Jun. 6, 2013
(86) PCT No.: PCT/IB2013/054661

§ 371 Date: Dec. 9, 2014

Related U.S. Application Data
(60) Provisional application No. 61/658,091, filed on Jun. 11, 2012.

(51) Int. Cl. A61F 5/56; A61F 5/042

(52) U.S. Cl. CPC A61F 5/56; A61F 5/042
USPC 128/845

(57) ABSTRACT

A neck extension device that positions a subject's chin away from the subject's upper chest lengthens the frontal area of the subject's neck and facilitates unobstructed breathing by opening the airway. According to some embodiments, the neck extension device includes a first surface and a second surface, where the first surface is configured to contact an area of the subject's head (e.g., the subject's mandible) and the second surface is configured to contact an area of subject's upper chest. The neck extension device may comprise a body member, which includes the first surface and the second surface on generally opposing sides. According to some embodiments, the neck extension device is configured to removably attach to the subject's neck.

### CROSS-REFERENCE TO RELATED APPLICATIONS

This patent application claims the priority benefit under 35 U.S.C. §119(e) of U.S. Provisional Application No. 61/658,091, filed on Jun. 11, 2012, the contents of which are herein incorporated by reference.

### BACKGROUND OF THE INVENTION

1. Field of the Invention

The present disclosure pertains to treating breathing disorders, and, in particular, to extending the neck of a subject to enhance air flow during breathing.

2. Description of the Related Art

Sleep apnea and other breathing disorders are affected by throat and neck structure, mainly by muscles and skin collapsing or relaxing internally and thereby blocking or limiting air flow. One method of opening the airway is by using a positive air pressure (PAP) device. Another method of expanding the neck/throat area is by applying negative pressure to the outer portion of the neck.

### SUMMARY OF THE INVENTION

Accordingly, it is an object of one or more embodiments of the present invention to provide a neck extension device. The neck extension device comprises a first surface and a second surface. The first surface is configured to contact an area of a subject's head. The second surface is configured to contact an area of the subject's upper chest. The first surface and the second surface are arranged such that, during use, the subject's chin is positioned away from the area of the subject's upper chest so as to lengthen the frontal area of the subject's neck and facilitate unobstructed breathing by opening the airway.

It is yet another aspect of one or more embodiments of the present invention to provide a method for extending the neck of a subject to enhance air flow during breathing. The method comprises (1) contacting an area of a subject's head with a first surface of a neck extension device; (2) contacting an area of the subject's upper chest with a second surface of the neck extension device; and (3) arranging the first surface and the second surface such that the subject's chin is positioned away from the area of the subject's upper chest so as to lengthen the frontal area of the subject's neck and facilitate unobstructed breathing by opening the airway.

It is yet another aspect of one or more embodiments to provide a neck extension device comprising first means, second means, and third means. The first means is for contacting an area of a subject's head. The second means is for contacting an area of the subject's upper chest. The third means is for arranging the first means and the second means such that the subject's chin is positioned away from the area of the subject's upper chest so as to lengthen the frontal area of the subject's neck and facilitate unobstructed breathing by opening the airway.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIGS. 1A and 1B illustrate different neck positions of a subject;

FIG. 2 illustrates a neck extension device in use by a subject, in accordance with one or more embodiments;

FIGS. 3A, 3B, 3C, and 3D illustrate different embodiments of a neck extension device; and

FIG. 4 illustrates a method for extending the neck of a subject to enhance air flow during breathing, in accordance with one or more embodiments.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

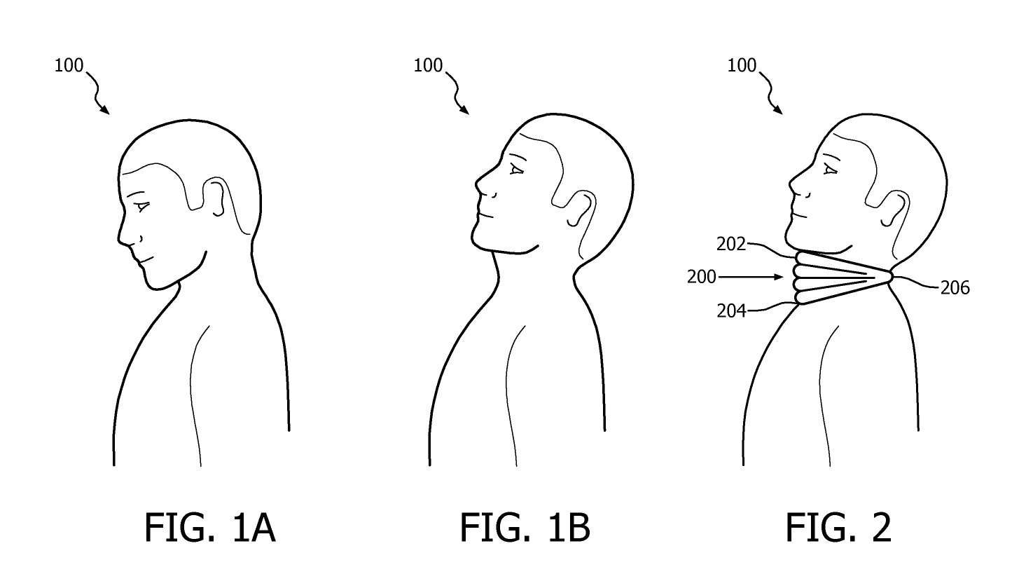

Exemplary embodiments provide a neck extension device that positions a subject's chin away from the subject's upper chest so as to lengthen the frontal area of the subject's neck and facilitate unobstructed breathing by opening the airway. FIGS. 1A and 1B illustrate different neck positions of a subject 100. In FIG. 1A, the head of subject 100 is tilted downward, thereby compressing and/or constricting the airway. In FIG. 1B, the head of subject 100 is extended upward, thereby elongating and/or dilating the airway. By replacing conventional breathing disorder treatments with a neck extension device, several disadvantages may be overcome. For example, as an alternative to PAP devices, a neck extension device used according the present disclosure eliminates inter alia a power source, a possibility of device failure due to electrical malfunction, sealing requirements for masks, headgear straps, and tubing of a breathing circuit. This may result in improved mobility and comfort, and enhanced ease of transport and storage.

FIG. 2 illustrates a neck extension device 200 in use by subject 100, in accordance with one or more embodiments. Neck extension device 200 includes a first surface 202 and a second surface 204. First surface 202 is configured to contact an area of subject 100's head. The area of subject 100's head contacted by first surface 200 may include subject 100's mandible, forehead, and/or other area. Second surface 204 is configured to contact an area of subject 100's upper chest. First surface 202 and second surface 204 are arranged such that, during use, subject 100's chin is positioned away from the area of subject 100's upper chest so as to lengthen the frontal area of subject 100's neck and to facilitate unobstructed breathing by opening the airway.

According to some embodiments, neck extension device 200 comprises a body member 206, which includes first surface 202 and second surface 204 on generally opposing sides. According to various embodiments, body member 206 physically or mechanically couples first surface 202 with second surface 204. As such, body member 206 may flexible or rigid, and fixed or adjustable. In some embodiments, body member 206 is self-adjusting, such as by spring force or memory. Neck extension device 200 and/or body member 206 may be formed by a single material or several composite materials and/or components. First surface 202 and/or second surface 204 maybe configured to conform to subject 100's body shape.

Body member 206 of neck extension device 200 may provide support for subject 100's neck. The support provided, however, may differ from conventional neck braces and/or other existing neck supports. For example, body member 206 may provide support preventing subject 100's head from tilting forward from a position where the neck is extended, but, at the same time, allow head rotation and/or lateral tilting of the head. In some embodiments, such as those where body member 206 is formed by foam, the lateral portions of body member 206 may have a lower resiliency relative to the anterior portion of body member 206. The anterior portion of body member 206 may be thicker or taller relative to the posterior portion to provide a neck position where the airway is opened.

According to some embodiments, neck extension device 200 is configured to removably attach to the subject's neck. Neck extension device 200 may have an open back with one or more closures (not depicted) configured to close body member 206 around subject 100's neck. Examples of such closures include snaps, buttons, hook-and-loop fastener (e.g., Velcro™), and/or other closure mechanisms. In one embodiment, body member 206 of neck extension device 200 is flexible such that neck extension device 200 can be stretched over subject 100's head in order to position neck extension device 200 around subject 100's neck.

In some embodiments, neck extension device 200 does not wrap entirely around subject 100's neck. Instead, neck extension device 200 may act as a chin rest. In such embodiments, neck extension device 200 may be secured by a garment worn by subject 100 and/or otherwise held in a fixed or semi-fixed position relative to subject 100's body. Neck extension device 200 may be a block and/or wedge the rests upon subject 100 neck, upper chest, and/or shoulders while subject 100 is in a prone position.

Some embodiments may include further features. By way of non-limiting example, one or more surfaces of neck extension device 200 (e.g., first surface 202 and/or second surface 204) may include an outer layer configured for breathability and/or comfort. Such an outer layer may include a fabric and/or other material. Neck extension device 200, or portions thereof, may be ventilated (e.g., passively or actively) for breathability and/or comfort. Some embodiments may facilitate determining and/or ensuring a proper neck angle and/or head position, which may be based on a measured angle and/or on measured airway resistance as a function of head position.

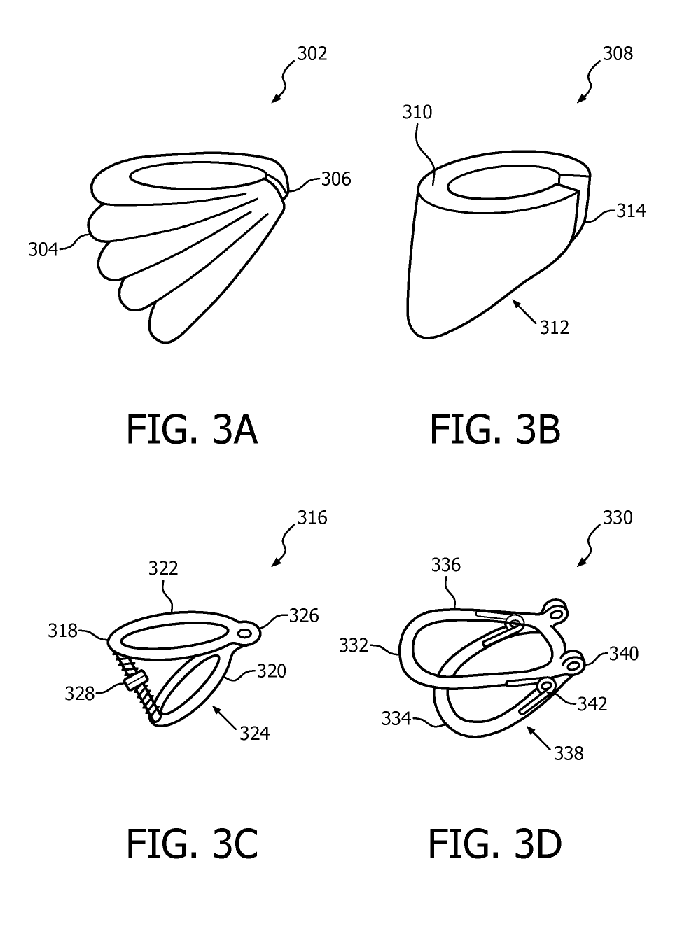

FIGS. 3A, 3B, 3C, and 3D illustrate different embodiments of a neck extension device (e.g., neck extension device 200). More specifically, FIG. 3A illustrates a bellows-type neck extension device 302, in accordance with one or more embodiments. Bellows-type neck extension device 302 may include one or more bellows 304. Individual bellows 304 may include an inflatable chamber. In some embodiments, the inflatable chamber is inflated by a gas, liquid, or other fluid. The pressure of the inflatable chamber may be adjustable. Such adjustments may be made oral inflation or by using an external pump (not depicted) that couples to bellows-type neck extension device 302 via a nozzle (not depicted) or other coupling device. According to some embodiments, bellows-type neck extension device 302 includes an internal pump that is operated electrically and/or manually. By increasing the pressure within bellows 304, the separation distance between subject 100's chin and chest will be increased, thereby further lengthening the frontal area of subject 100's neck. As depicted in FIG. 3A, bellows-type neck extension device 302 includes an open back 306, which facilitates removably attaching bellows-type neck extension device 302 to subject 100's neck. Open back 306 may be closed as described in connection with FIG. 2.

FIG. 3B illustrates a solid foam-type neck extension device 308, in accordance with one or more embodiments. Solid foam-type neck extension device 308 may be a generally annular structure having an overall wedge configuration. The annular structure of solid foam-type neck extension device 308 is configured to encompass subject 100's neck. Solid foam-type neck extension device 308 may be formed by a solid foam element shaped so that, when worn, a first surface 310 (upper surface) and a second surface 312 (lower surface) position subject 100's head and neck to lengthen the frontal area of subject 100's neck. That is, an anterior portion of the wedge configuration is thicker than a posterior portion of the wedge configuration. Solid foam-type neck extension device 308 may include an open back 314, which facilitates removably attaching solid foam-type neck extension device 308 to subject 100's neck. Open back 314 may be closed as described in connection with FIG. 2. In some embodiments, solid foam-type neck extension device 308 may secure to subject 100's neck in a cuffing manner where solid foam-type neck extension device 308 is flexible enough so that open back 314 can open around subject 100's neck to take it on or off, but securely closes when worn.

FIG. 3C illustrates a rigid adjustable-type neck extension device 316, in accordance with one or more embodiments. As depicted in FIG. 3C, rigid adjustable-type neck extension device 316 includes a first member 318 and a second member 320. First member 318 forms a first surface 322 configured to contact an area of subject 100's head, such as subject 100's mandible. Second member 320 forms a second surface 324 configured to contact an area of subject 100's upper chest. First member 318 and second member 320 may be coupled together via a hinge 326 and/or another coupling mechanism. An adjustable element 328 mechanically couples first member 318 with second member 320, and is configured to facilitate adjusting a separation distance between the anterior sides of first surface 322 and second surface 324 such that an angle between first surface 322 and second surface 324 is adjusted. In exemplary embodiments, the angle between first surface 322 and second surface 324 may fall in the range of about 90 to 180 degrees. The angle may depend on subject 100's anatomy. The angle may be determined based on the relative inclinations of subject 100's mandible and chest. Adjustable element 328 may include a screw-type mechanism and/or other mechanism suitable for adjusting length.

FIG. 3D illustrates a rigid adjustable-type neck extension device 330, in accordance with one or more embodiments. As depicted in FIG. 3D, rigid adjustable-type neck extension device 330 includes a first member 332 and a second member 334. First member 332 forms a first surface 336 configured to contact an area of subject 100's head, such as subject 100's mandible. Second member 334 forms a second surface 338 configured to contact an area of subject 100's upper chest. First member 332 and second member 334 may be coupled together via one or more hinges 340 and/or another coupling mechanism. One or more torsion springs 342 may be configured to provide a tensive force between first member 332 and second member 334 to provide resistance against lessening the angle between first member 332 and second member 334.

The embodiments depicted FIGS. 3A, 3B, 3C, and 3D are intended to be illustrative and are not limiting as other embodiments are contemplated and within the scope of the disclosure. For example, one or more features described in connection with FIGS. 3A, 3B, 3C, and/or 3D can be combined with, omitted from, and/or interchanged among various embodiments described herein.

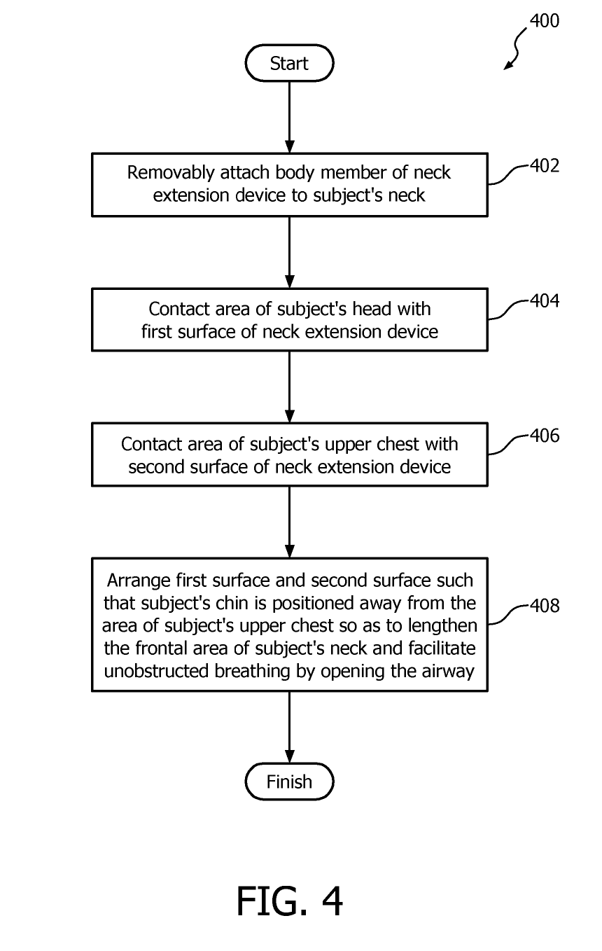

FIG. 4 illustrates a method 400 for extending the neck of a subject to enhance air flow during breathing, in accordance with one or more embodiments. The operations of method 400 presented below are intended to be illustrative. In some embodiments, method 400 may be accomplished with one or more additional operations not described, and/or without one or more of the operations discussed. Additionally, the order in which the operations of method 400 are illustrated in FIG. 4 and described below is not intended to be limiting.

At an operation 402, a body member (e.g., body member 206) of a neck extension device (e.g., neck extension device 200, bellows-type neck extension device 302, solid foam-type neck extension device 308, rigid adjustable-type neck extension device 316) is removably attached to a subject's (e.g., subject 100) neck. In exemplary embodiments, the body member includes a first surface (e.g., first surface 202, first surface 310, first surface 322) and a second surface (e.g., second surface 204, second surface 312, second surface 324) on generally opposing sides.

At an operation 404, an area of the subject's head is contacted with the first surface of the neck extension device. According to various embodiments, the area of the subject's head contacted by the first surface may include the subject's mandible, forehead, and/or other area.

At an operation 406, an area of the subject's upper chest is contacted with the second surface of the neck extension device.

At an operation 408, the first surface and the second surface are arranged such that the subject's chin is positioned away from the area of the subject's upper chest so as to lengthen the frontal area of the subject's neck and facilitate unobstructed breathing by opening the airway. In some embodiments, a separation distance between the first surface and the second surface is adjusted using an adjustable element (e.g., adjustable element 328) and/or by otherwise adjusting the neck extension device.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A neck extension device, comprising:
a first surface configured to contact an area of a subject's head; and
a second surface configured to contact an area of the subject's upper chest, and wherein the first surface and the second surface are arranged such that, during use, the subject's chin is positioned away from the area of the subject's upper chest so as to lengthen the frontal area of the subject's neck and facilitate unobstructed breathing by opening the airway, wherein a lateral portion of the neck extension device has a lower resiliency relative to an anterior portion of the neck extension device to allow head rotation, and/or lateral tilting of the head relative to the lateral portion and to prevent the subject's head from tilting forward from a position where the neck is extended in the anterior direction.

2. The neck extension device of claim 1, wherein the first surface and the second surface respectively form an upper surface and a lower surface of a generally annular structure having an overall wedge configuration, the annular structure being configured to encompass the subject's neck, an anterior portion of the wedge configuration being thicker than a posterior portion of the wedge configuration so as to lengthen the frontal area of the subject's neck.

3. The neck extension device of claim 1, further comprising an adjustable element mechanically coupling the first surface with the second surface, the adjustable element being configured to facilitate adjusting a separation distance between the anterior sides of the first surface and the second surface such that an angle between the first surface and the second surface is adjusted.

4. The neck extension device of claim 1, further comprising a body member, the body member including the first surface and the second surface on generally opposing sides, the body member being configured to removably attach to the subject's neck.

5. The neck extension device of claim 4, wherein the body member is formed by one or more of:
one or more bellows;
a solid foam element; or
an inflatable chamber.

6. A method for extending the neck of a subject to enhance air flow during breathing, the method comprising:
contacting an area of a subject's head with a first surface of a neck extension device;
contacting an area of the subject's upper chest with a second surface of the neck extension device;
arranging the first surface and the second surface such that the subject's chin is positioned away from the area of the subject's upper chest so as to lengthen the frontal area of the subject's neck and facilitate unobstructed breathing by opening the airway; and
arranging the resiliency of the neck extension device so that a lateral portion has a lower resiliency relative to an anterior portion to allow head rotation and/or lateral tilting of the head relative to the lateral portion and to prevent the subject's head from tilting forward from a position where the neck is extended in the anterior direction.

7. The method of claim 6, wherein the area of the subject's head contacted by the first surface includes the subject's mandible.

8. The method of claim 6, further comprising adjusting a separation distance between the anterior sides of the first surface and the second surface to adjust an angle therebetween using an adjustable element of the neck extension device mechanically coupling the first surface with the second surface.

9. The method of claim 6, further comprising removably attaching a body member of the neck extension device to the subject's neck, the body member including the first surface and the second surface on generally opposing sides.

10. The method of claim 9, wherein the body member is formed by one or more of:
one or more bellows;
a solid foam element; or
an inflatable chamber.

11. A neck extension device, comprising:
first means for contacting an area of a subject's head;
second means for contacting an area of the subject's upper chest;
third means for arranging the first means and the second means such that the subject's chin is positioned away from the area of the subject's upper chest so as to lengthen the frontal area of the subject's neck and facilitate unobstructed breathing by opening the airway; and
fourth means for arranging a lateral portion of the neck extension device with a lower resiliency relative to an anterior portion to allow head rotation and/or lateral tilting of the head relative to the lateral portion and to prevent the subject's head from tilting forward from a position where the neck is extended in the anterior direction.

12. The neck extension device of claim 11, wherein the first means and the second means respectively form an upper surface and a lower surface of a generally annular structure having an overall wedge configuration, the annular structure being configured to encompass the subject's neck, an anterior portion of the wedge configuration being thicker than a posterior portion of the wedge configuration so as to lengthen the frontal area of the subject's neck.

13. The neck extension device of claim 11, further comprising fourth means for adjusting a separation distance between the anterior sides of the first means and the second means such that an angle between the first means and the second means is adjusted.

14. The neck extension device of claim 11, further comprising fifth means for removably attaching the neck extension device to the subject's neck, the fifth means including the first means and the second means on generally opposing sides.

15. The neck extension device of claim 14, wherein the fifth means is formed by one or more of:
one or more bellows;
a solid foam element; or
an inflatable chamber.
